(19) United States
(12) Patent Application Publication
(10) Pub. No.: US 2006/0161027 A1
(43) Pub. Date: Jul. 20, 2006

(54) Method of producing 1, 3-propanediol

(75) Inventors: Toru Okoshi, Kanagawa (JP); Naoko Fujita, Kanagawa (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: MITSUBISHI CHEMICAL CORPORATION, Tokyo (JP)

(21) Appl. No.: 11/268,578

(22) Filed: Nov. 8, 2005

Related U.S. Application Data
(63) Continuation of application No. PCT/JP04/06422, filed on May 6, 2004.

(30) Foreign Application Priority Data
May 8, 2003 (JP) P. 2003-130643

(51) Int. Cl. C07C 29/74; C07C 31/18

(52) U.S. Cl. 568/868

(57) ABSTRACT

An object of the present invention is to provide a method of purification of 1,3-propanediol which gives less colored polytrimethylene ether glycol. The invention relates to a method of producing 1,3-propanediol which includes subjecting crude 1,3-propanediol having a purity of 95% by weight or greater to a heat treatment in the presence of a base, followed by distillation to give a distillate of purified 1,3-propanediol.

### FIELD OF THE INVENTION

The present invention relates a method of producing 1,3-propanediol. Particularly, the invention relates to a method of producing 1,3-propanediol which can give polytrimethylene ether glycol having a small Hazen color number when a dehydrative condensation reaction of 1,3-propanediol is allowed to make a polymer.

### BACKGROUND ART

One of important applications of 1,3-propanediol is production of polytrimethylene ether glycol. However, many of industrially available 1,3-propanediol often provide colored polytrimethylene ether glycol. Therefore, various methods have been proposed which enable less colored polytrimethylene ether glycol to be obtained through purification of 1,3-propanediol.

For example, a method in which 1,3-propanediol is treated in an aqueous acid solution, and thereafter a base is added to this aqueous acid solution to make the aqueous solution basic, followed by distillation (Patent Document 1). However, because purification is conducted as an aqueous solution in this method, amount of the treatment is increased to cause a problem of enlargement of the apparatus for the treatment. In addition, problem of a large amount of energy consumption may be caused because water and 1,3-propanediol are separated by distillation.

Also, a method was described in which 1,3-propanediol is subjected to a heat treatment in the presence of an acid catalyst, followed by distillation (Patent Document 2). In this method, a resin having a perfluorosulfonic acid group is used as an acid catalyst, although this reagent is expensive. In addition, high treatment temperature may lead to production of oligomer through dehydrative condensation of 1,3-propanediol, and a problem of low yield of 1,3-propanediol which can be recovered by distillation may be also caused.

Furthermore, a method in which a base is added to a fermented liquid containing 1,3-propanediol produced by a fermentation process to adjust the pH of 7 or higher, and the liquid is concentrated by heating, followed by separation of 1,3-propanediol from the fermented liquid by a process such as distillation or filtration (Patent Document 3).

However, the present inventors elucidated that the following problems may be raised in this method. More specifically, when the heating temperature is low, impurities in crude 1,3-propanediol are not sufficiently eliminated to exert unsatisfactory effect of purification. To the contrary, when the heating temperature is too high, decomposition reaction proceeds in crude 1,3-propanediol, resulting in contamination of impurities generated by the decomposition reaction in distilled 1,3-propanediol. Also, when greatly colored 1,3-propanediol is used as a basic ingredient to subject to distillation in the presence of a base, impurities in distilled 1,3-propanediol can not be eliminated enough.

In any of these cases, problems of coloring are involved when polytrimethylene ether glycol is produced by a dehydrative condensation reaction using distilled 1,3-propanediol.
[Patent Document 1]
U.S. Pat. No. 5,527,973
[Patent Document 2]
U.S. Pat. No. 6235948 B1
[Patent Document 3]
U.S. Pat. No. 6361983 B1

### DISCLOSURE OF THE INVENTION

Accordingly, the present invention intends to provide a method that permits purified 1,3-propanediol to be obtained efficiently and safely which provides less colored polytrimethylene ether glycol from crude 1,3-propanediol by a dehydrative condensation reaction.

Although analysis with gas chromatography of 1,3-propanediol having comparatively high purity produced by a chemical synthetic process detects peak of impurities in only a slight amount, yellow coloring is caused when a dehydrative condensation reaction thereof is allowed to produce a polymer. The present inventors focused attention to this event, and investigated on methods for obtaining 1,3-propanediol which enables suppression of coloring when it is made into a polymer, through further purifying predominantly crude 1,3-propanediol having comparatively high purity. Accordingly, a method for solving these problems was found.

Moreover, it was also found that safe distillation operation can be carried out and 1,3-propanediol which gives less colored polytrimethylene ether glycol can be obtained by limiting the operating range in distillation of 1,3-propanediol in the presence of a base, taking into account of thermal stability of the solution in the distillation pot. Accordingly, the present invention was accomplished.

More specifically, first aspect of the invention is a method of producing 1,3-propanediol, which comprises subjecting crude 1,3-propanediol having a purity of 95% by weight or greater to a heat treatment in the presence of a base, followed by distillation to give a distillate of purified 1,3-propanediol.

Second aspect of the invention is a method of producing 1,3-propanediol, which comprises subjecting crude 1,3-propanediol to a heat treatment in the presence of a base at a temperature of 110° C. or greater and 200° C. or less, followed by distillation to give a distillate of purified 1,3-propanediol.

Third aspect of the invention is a method of producing 1,3-propanediol, which comprises giving a distillate of purified 1,3-propanediol by distillation of crude 1,3-propanediol in the presence of a base, the distillation being carried out under the condition that satisfies the following formula (1):
T≦200−C (1)

in the formula (1), represents distillation temperature (° C.); and C represents concentration of the base (mol %).

Fourth aspect of the invention is a method of producing polytrimethylene ether glycol which comprises allowing a dehydrative condensation reaction of purified 1,3-propanediol obtained by the above production method, in the presence of an acid catalyst.

According to the method of the invention, 1,3-propanediol which can give less colored polytrimethylene ether glycol by a dehydrative condensation reaction can be produced.

### BEST MODE FOR CARRYING OUT THE INVENTION

Hereinaftrer, the present invention will be explained in detail.

(Crude 1,3-propanediol)

According to the present invention, crude 1,3-propanediol which gives colored polytrimethylene ether glycol upon a dehydrative condensation is subjected to a purification treatment.

Generally, crude 1,3-propanediol which gives polytrimethylene ether glycol having a Hazen color number defined by American Public Health Association (APHA) standard of 500 or greater when polymerized under a standard polymerization condition described later is subjected to a purification treatment.

Examples of such crude 1,3-propanediol include those produced by hydroformylation of ethylene oxide followed by hydrogenation, those produced by hydration of acrolein in the presence of an acid catalyst followed by hydrogenation, and the like.

Such crude 1,3-propanediol usually contains carbonyl compounds such as aldehyde and ketone, and acetal or ketal compounds of these carbonyl compounds in an amount of approximately 400 ppm. Further, 1,3-propanediol produced by a fermentation process may give comparatively less colored polytrimethylene ether glycol, however, such 1,3-propanediol produced by a fermentation process may be a subject of the treatment according to the invention when the quality thereof is deteriorated during storage so that it comes to give colored polytrimethylene ether glycol.

It is preferred that crude 1,3-propanediol has a low content of a component having a lower boiling point such as water or an organic solvent than 1,3-propanediol in order to reduce the burden in distillation described later.

Generally, crude 1,3-propanediol having a 1,3-propanediol content of 95% by weight or greater is subjected to a purification treatment.

It is preferred that 1,3-propanediol used in the purification treatment is less colored, usually having APHA of 100 or less, preferably 60 or less, and more preferably 30 or less.

According to the invention, crude 1,3-propanediol is first heated in the presence of a base. Because amount of impurities in the crude 1,3-propanediol is generally so slight that it is difficult to verify what change of impurities is caused by this heating, with analysis of 1,3-propanediol before and after the heat treatment. However, as is well-known, when a carbonyl compound is heated in the presence of a base, aldol condensation is caused to be converted into a compound having a greater molecular weight. Also in the invention, impurities which exist in the crude 1,3-propanediol and which are hardly separable from 1,3-propanediol by distillation may be converted into compounds having a greater molecular weight, which can be separated by distillation, by heating in the presence a base through aldol condensation.

(Base)

Examples of the base which may be used include alkali metal hydroxides such as sodium hydroxide, potassium hydroxide and caesium hydroxide;

alkaline earth metal hydroxides such as magnesium hydroxide, calcium hydroxide and barium hydroxide;

alkali metal carbonates and bicarbonates such as sodium carbonate, potassium carbonate, caesium carbonate, sodium bicarbonate and potassium bicarbonate;

basic carbonates such as basic magnesium carbonate;

carbonates of an alkaline earth metal such as calcium carbonate; alkoxides of an alkali metal such as sodium methylate and sodium ethylate;

alkali metal carboxylates such as sodium acetate and potassium acetate;

basic zeolites such as alumina carrying potassium fluoride, calcium fluoride and sodalite; and the like. Among them, preferred bases are hydroxides, carbonates and bicarbonates of an alkali metal, hydroxides of an alkaline earth metal and the like. In particular, sodium hydroxide, potassium hydroxide, sodium carbonate, potassium carbonate and calcium hydroxide are preferred in terms of low cost, favorable efficiency of treatment, and easy handling.

(Heat Treatment)

In the heat treatment of crude 1,3-propanediol in the presence a base, lower limit of the temperature is usually 80° C. or greater, preferably 110° C. or greater, more preferably 120° C. or greater, particularly preferably 130° C. or greater, and most preferably 140° C. or greater, while the upper limit thereof is 200° C. or less, preferably 190° C. or less, and more preferably 180° C. or less.

When this temperature is too low, effect of the treatment with a base may not be exerted to result in remaining impurities in 1,3-propanediol thereby giving colored polytrimethylene glycol. Additionally, a long period of the heat treatment time for exerting the effect of the treatment with a base tends to be necessary. To the contrary, too high temperature tends to result in coloring of the purified 1,3-propanediol, or give colored polytrimethylene glycol.

Grounds of these events when the temperature is too low are believed to be difficulties in proceeding of the condensation reaction of the carbonyl compound with the base, thereby leaving the residual carbonyl compound, or necessity of a long period of time for sufficient proceeding of the condensation reaction of the carbonyl compound. Alternatively, when the temperature is too high, it is speculated that decomposition reaction by 1,3-propanediol and the base is caused to lead to contamination of impurities in distillated 1,3-propanediol, as estimated from the analytical results by a differential scanning calorimeter described later.

Any pressure is permitted as long as 1,3-propanediol (boiling point: 213.5° C.) is kept in the liquid phase. Generally, the reaction is carried out at an ordinary pressure or an approximate pressure thereto.

The atmosphere is preferably an inert gas atmosphere such as nitrogen or argon so that quality of 1,3-propanediol is not deteriorated. If desired, the heat treatment may be conducted under a reduced pressure, or an inert gas maybe blown in crude 1,3-propanediol for accelerating elimination of impurities having a lower boiling point than 1,3-propanediol.

Time required for the treatment may vary depending on the heating temperature or the coexisting amount of the base, the lower limit is usually 0.5 hour or longer, preferably 1 hour or longer, and more preferably 1.5 hours or longer while the upper limit is usually 50 hours or less, preferably 20 hours or less, and more preferably 5 hours or less. When the time is too short, intended reaction such as aldol condensation reaction of the carbonyl compound may not proceed sufficiently to result in remaining impurities in 1,3-propanediol. Also, when the time is too long, burden on the purification of 1,3-propanediol may be increased.

The heat treatment may be conducted either in a batch-wise manner or in a continuous manner.

In connection with the amount of the base for use in the case of batch-wise manner, lower limit may be usually 0.0001 time by weight or greater, preferably 0.001 time by weight or greater of the crude 1,3-propanediol, while the upper limit is usually 0.3 time by weight or less, preferably 0.05 time by weight or less. Such an amount of the base may be added and heated while stirring.

In connection with the continuous manner, when an insoluble base is used as the base, any one of fixed bed flow process and suspended bed process can be employed. In case of the suspended bed process, crude 1,3-propanediol of 0.1 time by weight or greater, preferably 1 time by weight or greater, with the upper limit of usually 10⁵ times by weight or less, preferably 10³ times by weight or less of the suspended base per hour may be supplied to the suspended bed.

In case of the fixed bed flow process, crude 1,3-propanediol of 0.001 time by weight or greater, preferably 0.01 time by weight or greater, with the upper limit of 10⁵ times by weight or less, preferably 10³ times by weight or less of the base per hour may be supplied to the fixed bed.

The heating is preferably conducted such that purified 1,3-propanediol gives polytrimethylene ether glycol having a Hazen color number of 330 or less under the standard polymerization condition described later.

(Distillation)

The crude 1,3-propanediol after subjecting to the heat treatment is distilled to recover the distillate of purified 1,3-propanediol as a product. Impurities in 1,3-propanediol which colorize polytrimethylene ether glycol are converted by the heat treatment into compounds which can be readily separated from 1,3-propanediol by distillation, therefore, the distillation can be carried out with a simple distillation apparatus. Upper limit of the temperature upon distillation is usually not higher than the temperature of the heat treatment, preferably 190° C. or less, and more preferably 150° C. or less. Further, the lower limit is usually 40° C. or greater, and preferably 60° C. or greater. When the distillation temperature is too high, a decomposition reaction in crude 1,3-propanediol may be caused. Thus, impurities derived from the decomposition reaction may be contaminated in the distilled components, leading to tendency to result in colorization of polytrimethylene ether glycol. To the contrary, when the temperature is too low, burden for promoting degree of the reduced pressure for distillation of 1,3-propanediol may be increased. Upon distillation, partial neutralization of the base may be conducted beforehand, or the distillation may be carried out after conducting entire neutralization.

Moreover, it is preferred that low-boiling distillates are eliminated in the distillation, because amount of contamination of 2-hydroxylethyl-1,3-dioxane included as an impurity can be decreased (see, Reference Example 1, described later).

According to the invention, an amount of the low-boiling distillates to be eliminated is 1% byweight or less, preferably 4% by weight or less, and more preferably 10% by weight or less of 1,3-propanediol, per total distillates.

Also, upon distillation, limitation of the distillation temperature and concentration ratio of the base to fall within a specified range enables thermostability of the solution remaining in the distillation to be improved, thereby allowing safe distillation to be carried out.

In connection with thermostability of the solution remaining in the distillation pot (residual solution in the pot), information can be generally obtained by a differential scanning calorimeter (DSC), which decides the risk based on the temperature of initiation of exothermic heat, heating value, difference in temperatures between exothermic peaks and the like.

Because heating value of 100 J/g, as the magnitude of the exothermic peak, is believed to be one indication of risk, distillation in the temperature range to give accumulated heating value in DSC of the residual solution in pot being 100 J/g or less is preferred. However, when there is an exothermic peak adjacent to in the higher temperature region and the difference in temperature from the temperature upon initiation of heating is slight, the temperature may be elevated due to exothermic heat in the lower temperature range. Accordingly, the temperature reaches to that of initiation of exothermic heat in the higher temperature region to induce exothermic heat in the higher temperature region, thereby increasing the degree of risk. Therefore, the distillation operation is preferably carried out in the temperature range to generate low exothermic heat, for example, within the temperature range so that accumulated heating value of 50 J/g or less is provided.

Condition for operation on distillation employed in the invention preferably falls within the range to satisfy the following formula (1):
T≦200−C (1)

wherein T represents distillation temperature (° C.), and C represents base concentration (mol %).

More preferably, the operation is carried out under a condition to satisfy the following formula (2):
T≦180−0.8×C (2)

Distillation temperature herein means the temperature of the solution in the distillation pot. Also, base concentration referred to herein is represented by a concentration (mol %) of cationic component of the base in the solution in the distillation pot. When a salt of a week acid-strong base is used, it is represented by a concentration of the strong base cationic component (for example, in case of K₂CO₃, represented by concentration of total K⁺). When the base was neutralized after the heat treatment with a base, it is represented by a concentration of the cationic component excluding neutralized matter in case of neutralization with a strong acid, and it is represented by a concentration of total cationic components in case of neutralization with a week acid. Additionally, in case where the solution in the distillation pot is slurry, it is represented by a concentration of the base dissolved in the 1,3-propanediol solution in the distillation pot.

In the range of the higher temperature and in the range of the higher base concentration than those described above, great exothermic heat is found in the DSC analysis, and a side reaction such as a decomposition reaction is liable to be caused in the solution in the distillation pot.

With respect to grounds of this exothermic reaction in the DSC analysis, when a concentrated 1,3-propanediol solution including the base is compared with a mixture of 1,3-propanediol and the base having the same concentration, almost similar exothermic behavior was exhibited. Therefore, it is assumed to be predominantly caused by the action of 1,3-propanediol and the base.

When distillation in the presence of a base is carried out under the operation condition in the range of the temperature and base concentration higher than those described above, thermostability of the solution in the distillation pot becomes inferior, and the distillation temperature can not be controlled to bring high risk of a runaway state. Moreover, the solution in the distillation pot may be thermally decomposed, leading to contamination of impurities in the distillation component to give colored 1,3-propanediol, as the case may be. In addition, when 1,3-propanediol distilled under a condition falling within such operation ranges is used as a basic ingredient, colored polytrimethylene glycol is prone to be yielded.

Accordingly, when 1,3-propanediol is distilled under a condition falling within the aforementioned operation ranges, not only safe operation but also suppression of the decomposition reaction in the distillation pot will be enabled. Therefore, 1,3-propanediol with less impurities can be obtained to give less colored polytrimethylene glycol.

(Purified 1,3-propanediol)

Purified 1,3-propanediol obtained according to the invention gives significantly less colored polytrimethylene ether glycol than the case where crude 1,3-propanediol prior to purification is used as a basic ingredient. Specific degree of coloring may vary depending on selection of crude 1,3-propanediol to be subjected to purification, purification condition, as well as selection of the polymerization condition and the like. However, when polymerization is conducted under the following standard polymerization condition, crude polytrimethylene ether glycol having a Hazen color number of 330 or less, still more 200 or less, or purified polytrimethylene ether glycol can be readily obtained. When purification operation is employed after the polymerization, Hazen color number will be hardly changed between crude polytrimethylene ether glycol and purified polytrimethylene ether glycol.

Production of polytrimethylene ether glycol under standard polymerization condition herein means the following method.

Standard Polymerization Condition:

Into a 100 ml four-necked flask equipped with a distillation tube, a nitrogen introducing tube, a thermometer and a stirrer is charged 50.0 g of 1,3-propanediol while supplying nitrogen at a rate of 100 Nml/min. Thereto is slowly added 0.697 g of 95% by weight conc. sulfuric acid while stirring. The flask is placed in an oil bath, and heated to 155° C. After elevating the temperature in about 30 min to adjust the liquid temperature of 155° C.±2° C. and keeping it for 9 hrs to permit the reaction, the flask is left to stand at room temperature to allow cooling. Water generated during the reaction is distilled away together with nitrogen. Thus resulting product is used as crude polytrimethylene ether glycol.

The crude polytrimethylene ether glycol (mixture) obtained under the above standard polymerization condition is purified by a common procedure.

The purification operation may be carried out by, for example, the following operation (purification operation 1).

The reaction liquid cooled to room temperature is transferred into a 300 ml egg plant-shaped flask using 50 g of n-butanol, and thereto is added 50 g of desalted water, followed by mild reflux for 1 hour to execute hydrolysis of sulfate ester. After cooling to room temperature by leaving to stand, the lower layer (water layer) among two layers separated is removed. To the upper layer (oil layer) is added 0.5 g of calcium hydroxide followed by stirring at room temperature for 1 hour, and the mixture is heated to 60° C. Then n-butanol and water are distilled away under a reduced pressure. Thus resulting oil layer is dissolved in 150 g of n-butanol, and filtrated with a 0.45 μm filter to eliminate insoluble matter. The filtrate is heater to 60° C., and n-butanol is distilled away under a reduced pressure. Thus resulting oil layer is dried in vacuo for 6 hours to obtain purified polytrimethylene ether glycol.

(Method of Producing Polytrimethylene Ether Glycol)

Polytrimethylene ether glycol can be produced by allowing a dehydrative condensation reaction, in the presence of a catalyst, of 1,3-propanediol obtained according to the method of production of the invention.

Examples of the catalyst which may be used in the production of polytrimethylene ether glycol include arbitrary acid catalysts which have been conventionally known to produce ether bonds by a dehydrative condensation reaction of alcoholic hydroxyl groups. Also, a base catalyst may be used together with the acid catalyst. The catalyst may be any one that acts as a homogenous catalyst through dissolution in the reaction system, or acts as a heterogeneous catalyst without dissolution.

Examples of the acid include heteropoly acids of sulfuric acid, phosphoric acid, fluorosulfuric acid, phosphotungstic acid and the like, alkylsulfonic acid which may be fluorinated at its alkyl chain such as methanesulfonic acid, trifluoromethanesulfonic acid, octanesulfonic acid and 1,1,2,2-tetrafluoroethanesulfonic acid, benzenesulfonic acid and benzenesulfonic acid which may have an alkyl side chain in the ring, arylsulfonic acid such as, for example, para-toluenesulfonic acid. Examples of the latter include metal complex oxide such as activated white earth, zeolite, silica-alumina and silica-zirconia, and resins having a perfluoroalkylsulfonic acid group as a side chain. Among these, sulfuric acid, phosphoric acid, benzenesulfonic acid, para-toluenesulfonic acid and the like are preferred in light of being readily available and inexpensive. Of these, sulfuric acid is most preferred.

Examples of the base catalyst which may be preferably used in combination include organic bases, and alkali metals. In particular, organic bases are preferred.

As the organic base, a nitrogen-containing organic base, particularly a nitrogen-containing organic base having a tertiary nitrogen atom is preferred. Illustrative examples thereof include nitrogen-containing heterocyclic compounds having a pyridine skeleton such as pyridine, picoline and quinoline, nitrogen-containing heterocyclic compounds having an N—C═N bond such as N-methylimidazole, 1,5-diazabicyclo[4.3.0]-5-nonene and 1,8-diazabicyclo[5.4.0]-7-undecene, trialkylamine such as triethylamine and tributylamine, and the like. Among them, those having a pyridine skeleton, nitrogen-containing heterocyclic compounds having an N—C═N bond are preferred, and pyridine is most preferred in light of being readily available and inexpensive.

When the acid and the base described above are used in combination, both compounds may be present in the reaction system independently, or may form a salt between the acid and the organic base. Alternatively, a compound previously forming a salt of the acid with the organic base may be also used.

Preferable examples of the alkali metal that is the base of the catalyst include Li, Na, K and Cs, and Na is particularly preferred. When the alkali metal is used, one forming an alkali metal salt between an alkali metal and an acid of the catalyst is preferably used.

Examples of the alkali metal salt include salts of mineral acid such as sulfate, hydrogen sulfate, halide, phosphate, hydrogen phosphate and borate, organic sulfonate such as trifluoromethanesulfonate, paratoluenesulfonate and methanesulfonate, carboxylate such as formate and acetate, and the like. In the reaction system, coexistence of the alkali metal salt and free acid is preferred. In this instance, it is preferred that the acid forming the alkali metal salt and the free acid are identical. In this instance, although the acid that is a catalyst and the alkali metal salt thereof may be used each, a catalyst including a desired acid and an alkali metal salt may be prepared by allowing a reaction of a carbonate, bicarbonate, hydroxide of an alkali metal or a metal alone with an acid that is the catalyst. For example, a reaction is allowed in a polyol that is a reaction substrate between alkali metal carbonate and sulfuric acid to give a solution containing sulfuric acid and an alkali metal salt of sulfuric acid.

The acid for the catalyst may be usually used in the range of 0.001 time by weight or greater and 0.3 time by weight or less of the polyol as a basic ingredient. In case of an acid that acts as a homogenous catalyst, it is preferably used in the range of 0.001 time by weight or greater and 0.1 time by weight or less. Also, when an acid that acts as a heterogeneous catalyst such as a resin having a perfluoroalkylsulfonic acid group as a side chain is used in a continuous reaction, a process in which the acid is retained in the reaction apparatus without removing together with the reaction liquid, and the basic ingredient polyol is continuously supplied thereto may be employed. In this instance, the basic ingredient polyol with the lower limit being usually 0.01 time by weight or greater and preferably 0.1 time by weight or greater, and the upper limit being usually 10000 times by weight or less and preferably 1000 times by weight or less of the acid that is usually retained in the reaction apparatus per hour may be supplied. In this instance, equivalent ratio of the base to the acid in the reaction apparatus may be decreased in a time dependent manner, therefore, the basic ingredient polyol may be supplied with the base as needed such that desired value of the equivalent ratio of the organic base to the acid is maintained.

Amount of the base may be less than equivalent of the acid of the catalyst in case of the organic base, i.e., the base may be used in a ratio of amount not to neutralize all of the acid of the catalyst. The base may be used to be preferably 0.01 equivalent or greater, more preferably 0.05 equivalent or greater, and preferably 0.9 equivalent or less, more preferably 0.5 equivalent or less of the acid to the catalyst.

In case of the alkali metal salt, it may be used to be, as an alkali metal, preferably 0.01 equivalent or greater, more preferably 0.05 equivalent or greater, and preferably 0.9 equivalent or less, more preferably 0.5 equivalent or less to the acid of the catalyst.

Production of polytrimethylene ether glycol by a dehydrative condensation reaction of 1,3-propanediol may be performed by either a batch-wise manner or a continuous manner. In case of the batch-wise manner, the basic ingredient polyol and the catalyst are charged in a reaction vessel, and the reaction may be allowed while stirring.

In case of the continuous reaction, for example, a process in which the basic ingredient polyol and the catalyst are continuously supplied into a reaction apparatus including serially connected numerous stirring baths or a flow type reaction apparatus, from one end, and the mixture is moved in the apparatus by a piston flow or a mode which is similar thereto, while the reaction liquid is continuously removed from another end. In case of the continuous reaction and where an acid that acts as a heterogeneous catalyst such as a resin having a perfluoroalkylsulfonic acid group as a side chain, a process in which the acid is allowed to retain in the reaction apparatus without removing together with the reaction liquid, to which the basic ingredient 1,3-propanediol is continuously supplied may be employed.

The dehydrative condensation reaction may be conducted at a temperature with the lower limit being usually 120° C. or greater, preferably 140° C. or greater, and the upper limit being usually 250° C., preferably at 200° C. or less. The reaction may be preferably conducted in an atmosphere of inert gas such as nitrogen or argon. The reaction pressure may fall within an arbitrary range as long as the reaction system can be kept being in a liquid phase, and the reaction is usually conducted under an ordinary pressure. If desired, in order to facilitate elimination of water generated by the reaction from the reaction system, the reaction may be conducted under a reduced pressure, or an inert gas may be circulated in the reaction system.

The reaction time may vary depending on using amount of the catalyst, reaction temperature, and desired yield and physical property for the produced dehydrative condensate, but the lower limit may be usually 0.5 hour or longer, preferably 1 hour or longer, while the upper limit may be usually 50 hours or less, preferably 20 hours or less. The reaction may be usually conducted in the absence of a solvent, however, if desired, a solvent may be also used. The solvent may be used through selecting ad libitum from organic solvents which have been used in common organic synthesis reactions, taking into account of vapor pressure under the reaction condition, solubility and stability of the raw materials. Examples of the organic solvent which may be used include e.g., aliphatic hydrocarbon compounds, aromatic hydrocarbon compounds and the like. These solvents may be substituted with a substituent such as an alkyl group, a halogen atom or the like. Moreover, boiling point of the organic solvent is preferably 120 to 300° C. An organic solvent that is azeotropic with water may be also used.

Separation and recovery of the produced polytrimethylene ether glycol from the reaction system can be conducted by a routine process. When an acid that acts as a heterogeneous catalyst is used, suspending acid is first removed from the reaction liquid by filtration or centrifugal separation. Then, aimed polytrimethylene ether glycol is obtained by eliminating low-boiling point oligomers and base by distillation or extraction of water and the like. When an acid that acts as a homogeneous catalyst is used, water is first added to the reaction liquid and layer splitting is allowed to give a polytrimethylene ether glycol layer and an aqueous layer containing the acid, base, oligomers and the like. In some cases, a part of the polytrimethylene ether glycol may form an ester with the acid used as the catalyst. In this case, water is added to the reaction liquid, and after heating to hydrolyze the ester, layer splitting is permitted. When an organic solvent having affinity to both polytrimethylene ether glycol and water is used with water in this operation, hydrolysis can be accelerated.

Furthermore, when polytrimethylene ether glycol has high viscosity and thus, operativity of the layer splitting is inferior, an organic solvent having affinity to polytrimethylene ether glycol, and being readily separable from polytrimethylene ether glycol by distillation may also be preferably used. Polytrimethylene ether glycol phase obtained by layer splitting is subjected to distillation to distil away the remaining water and organic solvent. Accordingly, aimed polytrimethylene ether glycol is obtained. When acid remains in the polytrimethylene ether glycol phase obtained by the layer splitting, the phase may be subjected to distillation after removing the remaining acid by washing with water or an aqueous alkali solution or by treating with an anion exchange resin or a solid base such as calcium hydroxide.

The polytrimethylene ether glycol obtained by the method of the invention has a weight average molecular weight (Mw) with lower limit being usually 600 or greater, preferably 1200 or greater, and the upper limit being usually 30000 or less, preferably 15000 or less, more preferably 10000 or less.

Number average molecular weight (Mn) has a lower limit of usually 500 or greater, preferably 1000 or greater, and an upper limit of usually 10000 or less, preferably 5000 or less.

Molecular weight distribution (Mw/Mn) is preferably as approximate to 1 as possible, with the upper limit being usually 3 or less, and preferably 2.5 or less.

Polytrimethylene ether glycol can be used in various applications such as elastic fibers, thermoplastic polyester elastomers, thermoplastic polyurethane elastomers, coating materials and the like.

### EXAMPLES

The present invention will be explained more specifically by way of Examples below, however, the invention is not limited by the following Examples unless it departs from the spirit thereof.

(Process of Calculating Yield)

Process for calculating yield in Examples 1 to 5 and Comparative Examples 1 to 2 is as in the following (“1,3-PD” represents 1,3-propanediol):
(weight of purified polytrimethylene glycol)*100/{weight of charged 1,3-PD-(charged 1,3-PD)*18/76}=yield (%)

Process for calculating yield in Examples 6 to 8 and Comparative Examples 3 to 5 is as in the following:
(yield/molecular weight calculated by NMR)×(polimerization degree calculated from NMR)=(number of moles of polymerized 1,3-PD);
(number of moles of polymerized 1,3-PD)/(number of moles of basic ingredient 1,3-PD)×100=yield (%)
(Molecular Weight and Molecular Weight Distribution)

Molecular weight of purified polytrimethylene ether glycol (Examples 1 to 5 and Comparative Examples 1 to 2) was measured by gel permeation chromatography under the following condition.
Column: TSK-GEL GMH_{XL}-N (7.8 mm ID×30.0 cm L) (Tosoh Corporation)
Weight calibration: POLYTETRAHYDROFURAN CALIBRATION KIT
   (Polymer Laboratories)

(Mp=547000, 283000, 99900, 67500, 35500, 15000, 6000, 2170, 1600, 1300)

Solvent: Tetrahydrofuran

Moreover, Mn and Mw mean as described below, respectively.

Mn: Number average molecular weight calculated by gel permeation chromatography (calculated using polytetrahydrofuran as a standard).

Mw: Weight average molecular weight calculated by gel permeation chromatography (calculated using polytetrahydrofuran as a standard).

Molecular weight of crude polytrimethylene glycolpolyether following the polymerize reaction (Example 6 and Comparative Examples 3 to 5) was measured by a nuclear magnetic resonance (NMR) method.

Sample was dissolved in chloroform-d (manufactured by ACROS ORGANICS, TMS 0.03 v/v%, 99.8+atm % D, lot: A018554501), and analyzed with a ¹H-NMR apparatus (manufactured by BRUKER, AVANCE400 (400 MHz)). Molecular weight was determined by the following formula as a value obtained when every sulfate ester generated was hydrolyzed (ppm based on TMS).
Molecular weight=[58×(methylene peak integration value at 1.8 ppm/2)/{(methylene peak integration value at 3.8 ppm+methylene peak integration value at 4.3 to 4.4 ppm)/4}]+18)
(Hazen Color Number)

Hazen color number was determined using a standard solution prepared by diluting an APHA color number standard solution (No. 500) manufactured by Kishida Chemical Co., Ltd., according to JIS K0071-1. As the color-difference meter, a calorimetric color-difference meter ZE-2000 manufactured by Nippon Denshoku Industries Co., Ltd. was employed, and measurement was conducted under a condition with a cell thickness of 10 mm.

### Example 1

Into a 200 ml four-necked flask equipped with a distillation tube, a nitrogen introducing tube, a thermometer and a stirrer were charged 100.0 g of 1,3-propanediol (reagent manufactured by Aldrich Co., purity: 98%, Batch #00312JO) and 0.5 g of sodium hydroxide while circulating nitrogen at 100 Nml/min. The flask was placed in an oil bath, and heated. When the liquid temperature reached to 147° C., the temperature was kept at 147 to 152° C. Two hours later, the flask was recovered, and left to stand to allow cooling to room temperature. Then, the mixture was subjected to simple distillation at about 100° C. under a reduced pressure. First drop of about 10 g was discarded, and the distillate of 81.2 g was recovered. The base remaining in the distillation pot then was entirely dissolved in 1,3-propanediol remaining in the distillation pot, and the concentration was about 10 mol %. This 1,3-propanediol was polymerized under a standard condition, and then purified according to the aforementioned purification operation 1. The results are shown in Table 1.

### Comparative Example 1

Treatment of 1,3-propanediol was conducted completely similarly to Example 1 except that 1.0 g of Nafion NR50 (7 to 9 mesh) that is a resin having a perfluorosulfonic acid group was used in stead of sodium hydroxide, and Nafion was eliminated before carrying out the distillation. Accordingly, 77.59 g of a distillate was recovered. This 1,3-propanediol was polymerized under a standard polymerization condition, and then purified according to the aforementioned purification operation 1. The results are shown in Table 1.

### Comparative Example 2

1,3-propanediol that is the same as one used in the purification treatment in Example 1 and Comparative Example 1 was polymerized neat without purification was polymerized under a standard polymerization condition, and then purified according to the aforementioned purification operation 1. The results are shown in Table 1.

### Example 2

Into a 200 ml four-necked flask equipped with a reflux condenser, a nitrogen introducing tube, a thermometer and a stirrer were charged 100.0 g of 1,3-propanediol (reagent manufactured by Aldrich Co., purity: 98%, Batch #00312JO) and 0.66 g of sodium carbonate in a nitrogen atmosphere. The flask was placed in an oil bath, and heated. When the liquid temperature reached to 147° C., the temperature was kept at 147 to 152° C. Two hours later, the flask was recovered, and left to stand to allow cooling to room temperature. Then, the mixture was subjected to simple distillation at about 100° C. under a reduced pressure. First drop of about 10 g was discarded, and the distillate of 81.6 g was recovered. The base remaining in the distillation pot then was not entirely dissolved in 1,3-propanediol remaining in the distillation pot. Concentration of sodium carbonate also including undissolved matter was about 5 mol %. Cation concentration also including undissolved matter was about 11 mol%.

Into a 100 ml four-necked flask equipped with a distillation tube, a nitrogen introducing tube, a thermometer and a stirrer was charged 50 g of this 1,3-propanediol while supplying nitrogen at 100 Nml/min. Thereto was slowly added 0.697 g of 95% by weight conc. sulfuric acid while stirring. The flask was placed in an oil bath, and heated to 155° C. After elevating the temperature in about 30 min to adjust the liquid temperature of 155° C.±2° C. and keeping it for 9 hrs to permit the reaction, the flask was left to stand at room temperature to allow cooling. Water generated during the reaction was distilled away together with nitrogen. The reaction liquid cooled to room temperature was transferred into a 300 ml egg plant-shaped flask using 50 g of tetrahydrofuran, and thereto was added 50 g of desalted water, followed by mild reflux for 1 hour to execute hydrolysis of sulfate ester. After cooling to room temperature by leaving to stand, the lower layer (water layer) among two layers separated was removed. To the upper layer (oil layer) was added 0.5 g of calcium hydroxide followed by stirring at room temperature for 1 hour. Thereafter, 50 g of toluene was added to the mixture, and heated to 60° C. Then tetrahydrofuran, water and toluene were distilled away under a reduced pressure. Thus resulting oil layer was dissolved in 100 g of toluene,.and filtrated with a 0.45 μm filter to eliminate insoluble matter. The filtrate was heater to 60° C., and toluene was distilled away under a reduced pressure. Thus resulting oil layer was dried in vacuo for 6 hours to obtain purified polytrimethylene ether glycol. The results are shown in Table 1.

### Example 3

Treatment of 1,3-propanediol was conducted completely similarly to Example 2 except that 0.93 g of calcium hydroxide was used in stead of sodium carbonate. Accordingly, 80.8 g of a distillate was recovered. The base remaining in the distillation pot then was not entirely dissolved in 1,3-propanediol remaining in the distillation pot. Concentration of calcium hydroxide also including undissolved matter was about 9 mol %.

This 1,3-propanediol was polymerized completely similarly to Example 2, and the results are shown in Table 1.

### Example 4

Treatment of 1,3-propanediol was conducted completely similarly to Example 2 except that 0.86 g of potassium carbonate was used in stead of sodium carbonate. Accordingly, 74.6 g of a distillate was recovered. The base remaining in the distillation pot then was entirely dissolved in 1,3-propanediol remaining in the distillation pot. Concentration of potassium carbonate then was about 3 mol %, and the base concentration (concentration of cationic component) was about 6 mol %.

This 1,3-propanediol was polymerized completely similarly to Example 2, and the results are shown in Table 1.

### Example 5

Treatment of 1,3-propanediol was conducted completely similarly to Example 2 except that 0.70 g of potassium hydroxide was used in stead of sodium carbonate. Accordingly, 82.6 g of a distillate was recovered. The base remaining in the distillation pot then was entirely dissolved in 1,3-propanediol remaining in the distillation pot. The base concentration then was about 11 mol %.

This 1,3-propanediol was polymerized completely similarly to Example 2, and the results are shown in Table 1.

TABLE 1

| | Yield of | | | | |
|---|---|---|---|---|---|
| | polymer (%) | Mw | Mn | Mw/Mn | APHA color number |
| Example 1 | 97 | 3523 | 1773 | 1.99 | 150 |
| Comparative | 96 | 3430 | 1700 | 2.02 | 350 |
| Example 1 | | | | | |
| Comparative | 96 | 3591 | 1733 | 2.07 | >500 |
| Example 2 | | | | | |
| Example 2 | 94 | 3693 | 1867 | 1.98 | 150 |
| Example 3 | 94 | 3452 | 1810 | 1.91 | 150 |
| Example 4 | 94 | 3505 | 1852 | 1.89 | 150 |
| Example 5 | 94 | 3617 | 1909 | 1.90 | 150 |

### Example 6

Into a 200 ml four-necked flask equipped with a reflux condenser, a nitrogen introducing tube, a thermometer and a stirrer were charged 100.6 g of 1,3-propanediol (reagent manufactured by Aldrich Co., purity: 98%, Batch #10508AB) and 0.67 g of potassium hydroxide (manufactured by JUNSEI CHEMICAL CO., LTD., special grade, purity: 85% or greater, lot: 2E1459) in a nitrogen atmosphere. The flask was placed in an oil bath, and heated. After the inner temperature of the reaction vessel reached to 150° C., it was kept for two hours to perfect a heat treatment. The flask was recovered, and left to stand to allow cooling to room temperature. Then, the mixture was subjected to simple distillation at about 100° C. of the inner temperature of the reaction vessel under a reduced pressure. First drop of about 10 g was discarded, and the distillate of 68.2 g was recovered. The base concentration upon the heat treatment was 0.9 mol %, and the base concentration after completing the distillation was about 4 mol %.

Into a 100 ml four-necked flask equipped with a distillation tube, a nitrogen introducing tube, a thermometer and a stirrer incubated at 100° C. with a tape heater was charged 50 g of this 1,3-propanediol while supplying nitrogen at 100 Nml/min. Thereto was slowly added 0.697 g of 95% by weight conc. sulfuric acid while stirring. The flask was placed in an oil bath, and heated to allow the inner temperature of the reaction vessel to reach to 165° C. in about 30 min. Thereafter, it was kept heating and stirring to permit the reaction for 7 hours. The flask was left to stand at room temperature to allow cooling. Water generated during the reaction was distilled away together with nitrogen. The molecular weight calculated by NMR, and AHPA color number measured by a color-difference meter are shown in Table 2.

### Comparative Example 3

Heating and distillation of 1,3-propanediol were conducted completely similarly to Example 6 except that 101.1 g of 1,3-propanediol and 0.66 g of potassium hydroxide were used, and the temperature of the oil bath was set to be 235° C. and the temperature of the heat treatment of 1,3-propanediol was set to be 203° C. Accordingly, 70.0 g of a distillate was recovered. This 1,3-propanediol was polymerized completely similarly to Example 6, and the results are shown in Table 2. The base concentration was 0.9 mol % upon heat treatment, and was about 4 mol % when the distillation was completed.

### Comparative Example 4

Heating and distillation of 1,3-propanediol were conducted completely similarly to Example 6 except that 100.7 g of 1,3-propanediol and 0.72 g of potassium hydroxide were used, and the temperature of heat treatment of 1,3-propanediol was set to be 70° C. Accordingly, 68.0 g of a distillate was recovered. This 1,3-propanediol was polymerized completely similarly to Example 6, and the results are shown in Table 2.

### Reference Example 1

To 250 g of 1,3-propanediol (manufactured by Aldrich Co., 10508AB) was added 1.75 g of potassium hydroxide. The mixture was subjected to the heat treatment and distillation similarly to Example 1. For each of 10 g of initially effluent 1,3-propanediol (4% in charged 1,3-propanediol) and 220 g of main distillate (88% in charged 1,3-propanediol), a 20 wt % THF (manufactured by Kishida Chemical Co., Ltd., special grade, containing 0.03 wt % BHT) solution was prepared, which was analyzed under the GC condition described below. Ratio of the area of 2-hydroxylethyl-1,3-dioxane to the area of 1,3-propanediol was 0.2% in the former, and was 0.08% in the latter.

It is believed that 2-hydroxylethyl-1,3-dioxane is converted into 3-hydroxy propanone in the presence of water and the acid catalyst, to be a substance accounting for coloring. Therefore, removal of 2-hydroxylethyl-1,3-dioxane as much as possible will be efficacious in the sense of prevention of coloring.

GC Analytical Condition

Column: HR-20M; film thickness 0.25 μm,

0.25 mm ID×30 m

Carrier: nitrogen about 1.5 ml/min,

split ratio about 40

Oven temperature: 50° C.—(temperature elevation:

10° C./min)—230° C. (kept for 10 min)

Temperature of inlet and detector: 240° C.

### Comparative Example 5

Into a 1 L four-necked flask equipped with a reflux condenser, a nitrogen introducing tube, a thermometer and a stirrer were charged 700 g of 1,3-propanediol (reagent manufactured by Aldrich Co., purity: 98%, Batch #04427AB) and 4.9 g of potassium hydroxide in a nitrogen atmosphere. The flask was placed in an oil bath, and heated. After the inner temperature of the reaction vessel reached to 150° C., it was kept for two hours to perfect a heat treatment. The flask was recovered, and left to stand to allow cooling to room temperature. Then, the mixture was subjected to simple distillation at about 100° C. of the inner temperature of the reaction vessel under a reduced pressure. Entirety was recovered without discarding low-boiling distillates. Thus, the distillate of 67.5 g was recovered. This 1,3-propanediol was polymerized completely similarly to Example 6, and the results are shown in Table 2.

TABLE 2

| | Temperature of | Molecular weight | |
|---|---|---|---|
| | heat treatment of | calculated by | APHA color |
| | 1,3-propanediol(° C.) | NMR | number |
| Example 6 | 150 | 1633 | 320 |
| Comparative | 203 | 1650 | 470 |
| Example 3 | | | |
| Comparative | 70 | 1865 | 420 |
| Example 4 | | | |
| Comparative | 150 | 1771 | ≧500 |
| Example 5 | | | |

(Thermal Analysis Example of Remaining Liquid in Pot by Differential Scanning Calorimeter (DSC))

For DSC analysis in the following Examples, measurement was conducted under the condition described below.

Measurement apparatus: Seiko Instruments, Inc., DSC-6200

Calibration Method:

SUS sealed cell manufactured by Seiko Instruments, Inc. (made of SUS304, volume (15 μl)) used

Temperature and calorie both calibrated on 4 kinds of metals of In, Sn, Pb and Zn

Sample vessel: SUS sealed cell manufactured by Seiko Instruments, Inc. (made of SUS304, volume (15 μl))

Sampling atmosphere: Sampling carried out in an atmosphere sufficiently substituted with dry nitrogen (purity: 99.999% or greater, dew point: −60° C.)

Sample amount: about 2 mg

Measurement temperature: 30 to 500° C.

Elevation of temperature: 10° C./min

Measurement atmosphere: nitrogen (purity: 99.999% or greater, dew point: −60° C.)

### Example 7

Into a 300 ml four-necked flask equipped with a distillation tube, a nitrogen introducing tube, a thermometer and a stirrer were charged 150.0 g of 1,3-propanediol (reagent manufactured by Aldrich Co., purity: 98%, Batch #10508AB) and 1.07 g of potassium hydroxide in a nitrogen atmosphere. The flask was placed in an oil bath, and heated. When the liquid temperature reached to 147° C., the temperature was kept at 147 to 152° C. Two hours later, the flask was recovered, and left to stand to allow cooling to room temperature. Then, the mixture was subjected to simple distillation at about 100° C. under a reduced pressure. First drop of 6.7 g was discarded, and the distillate of 127 g was recovered. Residue in the pot then was 13.38 g when residual KOH matter was excluded, and the base concentration was 10 mol %.

DSC analysis performed on this residual liquid in the pot revealed an exothermic peak, and accumulated heating value upto about 240° C. was 100 J/g. Thus, risk of leading to a runaway reaction was suggested when distillation was carried out at a temperature not lower than this temperature. In addition, the temperature at which the accumulated heating value of 50 J/g is developed is about 190° C. Accordingly, it was verified that for safer distillation, to employ a temperature not higher than this temperature is preferred.

### Example 8

Similarly to Example 7, 1,3-propanediol was subjected to the base treatment and distillation. Samples were taken at the time point when the amount of the 1,3-propanediol liquid remaining in the distillation pot became 30% of the initial value, and at the time point when the same amount became about 5% or less of the initial value. These samples were subjected to the DSC analysis.

The base concentration at the time point when 30% of the initial value was attained was about 3 mol %, and the base concentration at the time point when about 5% or less of the initial value was attained was 16 mol % or greater.

In the former case, accumulated heating value to approximately 250° C. was 100 J/g, and in the latter case, still greater exothermic peak was observed, and the heating value to approximately 200° C. became 100 J/g. Thus, risk of leading to a runaway reaction was found when distillation was carried out at a temperature not lower than this temperature.

In addition, the temperature at which the accumulated heating value of 50 J/g is developed is 200° C. in the former case, and is 170° C. in the latter case. Accordingly, it was verified that for carrying out safer distillation, to employ a temperature not higher than this temperature is preferred.

Although the invention was explained in detail with reference to specific modes thereof, a variety of possible alterations and modifications without departing from the purport and scope of the invention will be apparent to persons skilled in the art.

The present application is based on Japanese Patent Application filed on May 8, 2003 (Japanese Patent Application No. 2003-130643), the entire contents of which are incorporated herein by reference.

### INDUSTRIAL APPLICABILITY

According to the present invention, a method that enables purified 1,3-propanediol to be obtained efficiently and safely which gives less colored polytrimethylene ether glycol from crude 1,3-propanediol by a dehydrative condensation reaction can be provided.

1. A method of producing 1,3-propanediol, which comprises subjecting crude 1,3-propanediol having a purity of 95% by weight or greater to a heat treatment in the presence of a base, followed by distillation to give a distillate of purified 1,3-propanediol.

2. A method of producing 1,3-propanediol, which comprises subjecting crude 1,3-propanediol to a heat treatment in the presence of a base at a temperature of 110° C. or greater and 200° C. or less, followed by distillation to give a distillate of purified 1,3-propanediol.

3. The method of producing 1,3-propanediol according to claim 1, wherein the heating is conducted in the presence of a base to 140° C. or greater and 200° C. or less.

4. The method of producing 1,3-propanediol according to claim 1, wherein the heating is conducted in the presence of a base to 140° C. or greater and 180° C. or less.

5. The method of producing 1,3-propanediol according to claim 1, wherein the heat treatment time is one hour or longer and 20 hours or less.

6. The method of producing 1,3 -propanediol according to claim 1, wherein the reaction is conducted in a batch-wise manner, and the amount of the base is 0.0001 time by weight or greater and 0.3 time by weight or less of 1,3-propanediol.

7. The method of producing 1,3 -propanediol according to claim 1, wherein the reaction is conducted in a continuous manner, and the amount of the base is 0.001 time by weight or greater and 105 times by weight or less of 1,3-propanediol per hour.

8. The method of producing 1,3-propanediol according to claim 1, wherein the base is selected from alkali metal hydroxide, alkaline earth metal hydroxide, alkali metal carbonate, alkali metal bicarbonate, carbonate of an alkaline earth metal, alkoxide of an alkali metal, alkali metal carboxylate and basic zeolite.

9. The method of producing 1,3-propanediol according to claim 1, wherein the distillation is carried out at a temperature not higher than the heating temperature in the presence of the base.

10. The method of producing 1,3-propanediol according to claim 1, wherein distillation is carried out at 150° C. or less.

11. The method of producing 1,3-propanediol according to claim 1, wherein 1% by weight or greater of low-boiling distillates per total distillates are eliminated upon carrying out distillation.

12. The method of producing 1,3-propanediol according to claim 1, wherein crude 1,3-propanediol gives polytrimethylene ether glycol having a Hazen color number of 500 or greater under a standard polymerization condition.

13. The method of producing 1,3-propanediol according to claim 1, wherein the heat treatment in the presence of a base is conducted so that purified 1,3-propanediol gives polytrimethylene ether glycol having a Hazen color number of 330 or less under a standard polymerization condition.

14. A method of producing 1,3-propanediol, which comprises giving a distillate of purified 1,3-propanediol by distillation of crude 1,3-propanediol in the presence of a base, said distillation being carried out under the condition that satisfies the following formula (1):
T≦200−C (1)
in the formula (1), T represents distillation temperature (° C.) ; and C represents concentration of the base (mol %).

15. A method of producing polytrimethylene ether glycol, which comprises allowing a dehydrative condensation reaction of the purified 1,3-propanediol obtained by the production method according to claim 1, in the presence of an acid catalyst.
